(19) United States
(12) Patent Application Publication
(10) Pub. No.: US 2024/0110149 A1
(43) Pub. Date: Apr. 4, 2024

(54) CLOSTRIDIUM AUTOETHANOGENUM WITH ENHANCED GROWTH RATE

(71) Applicant: Alliance for Sustainable Energy, LLC, Golden, CO (US)

(72) Inventors: Leah Renae FORD, Medord, MA (US); Michael Thomas GUARNIERI, Denver, CO (US); Michael G. RESCH, Golden, CO (US); Lauren Rae MAGNUSSON, Lakewood, CO (US)

(21) Appl. No.: 18/478,608

(22) Filed: Sep. 29, 2023

Related U.S. Application Data
(60) Provisional application No. 63/412,137, filed on Sep. 30, 2022.

(51) Int. Cl. C12N 1/20; C12N 9/02; C12P 7/06

(52) U.S. Cl. CPC C12N 1/20; C12N 9/0093; C12P 7/065; C12R 2001/145

(57) ABSTRACT

Disclosed herein are non-naturally occurring C. autoethanogenum adapted to electrolyzer off-gas feedstock conditions that possess increased growth rates when compared to naturally occurring naturally occurring C. autoethanogenum.

### CROSS-REFERENCE TO RELATED APPLICATIONS

This application claims the benefit of U.S. Patent Application No. 63/412137 filed on 30 Sep. 2022 which is incorporated by reference herein in its entirety.

### CONTRACTUAL ORIGIN

The United States Government has rights in this invention under Contract No. DE-AC36-08G028308 between the United States Department of Energy and the Alliance for Sustainable Energy, LLC, the Manager and Operator of the National Renewable Energy Laboratory.

### BACKGROUND

The biocatalyst, Clostridium autoethanogenum is an obligate anaerobe with native machinery capable of autotrophic conversion of carbon monoxide and carbon dioxide to ethanol and butanediol. C. autoethanogenum is a difficult organism to cultivate, with long lag times, and very narrow redox requirements. C. autothanogenum does not grow optimally under gas concentrations supplied by electrolyzers.

### SUMMARY

In an aspect, disclosed herein are non-naturally occurring C. autoethanogenum adapted to electrolyzer off-gas feedstock conditions that possess increased growth rates when compared to naturally occurring naturally occurring C. autoethanogenum.

In an aspect, disclosed herein is a non-naturally occurring C. autoethanogenum that when provided feedstock comprising carbon monoxide or carbon dioxide from electrolyzer off-gas or flue gas feedstock has improved growth rates and improved alcohol or acetate production rates when compared to a naturally occurring C. autoethanogenum. In an embodiment, the non-naturally occurring C. autoethanogenum comprises a mutation in at least one gene encoding for a Crp/Fnr family transcriptional regulator. In an embodiment, the non-naturally occurring C. autoethanogenum comprises a mutation that creates a stop codon in a Crp/Fnr family transcriptional regulator. In another embodiment, the non-naturally occurring C. autoethanogenum comprises a mutation in at least one gene encoding for a xanthine dehydrogenase family protein molybdopterin-binding subunit. In yet another embodiment, the non-naturally occurring C. autoethanogenum comprises a mutation in at least one gene encoding for a sporulation transcription factor. In an embodiment, the non-naturally occurring C. autoethanogenum comprises a mutation in sporulation transcription factor Spo0A. In an embodiment, the non-naturally occurring C. autoethanogenum comprises a mutation in at least one gene encoding for a transcription antitermination factor. In an embodiment, the non-naturally occurring C. autoethanogenum comprises a deletion of about 400 bp. In an embodiment, the non-naturally occurring C. autoethanogenum comprises a mutation in a nusB gene. In an embodiment, the non-naturally occurring C. autoethanogenum comprises a deletion of about 2.7 Kpb in deoB. In an embodiment, the non-naturally occurring C. autoethanogenum comprises a deletion of about 2.7 Kpb in CAETHG_RS19245. In an embodiment, the non-naturally occurring C. autoethanogenum produces ethanol.

In an aspect, disclosed herein is a method for making a bioderived fuel comprising providing a non-naturally occurring C. autoethanogenum feedstock comprising carbon monoxide or carbon dioxide from electrolyzer off-gas or flue gas feedstock and isolating an alcohol that is used to make the bioderived fuel. In an embodiment, the non-naturally occurring C. autoethanogenum comprises a mutation in at least one gene encoding for a xanthine dehydrogenase family protein molybdopterin-binding subunit. In an embodiment, the non-naturally occurring C. autoethanogenum comprises a mutation in at least one gene encoding for a Crp/Fnr family transcriptional regulator. In yet another embodiment, the non-naturally occurring C. autoethanogenum comprises a mutation in at least one gene encoding for a sporulation transcription factor. In an embodiment, the non-naturally occurring C. autoethanogenum comprises a mutation in at least one gene encoding for a transcription antitermination factor. In an embodiment, the non-naturally occurring C. autoethanogenum comprises a mutation in a nusB gene. In an embodiment, the non-naturally occurring C. autoethanogenum comprises a deletion of about 2.7 Kpb in deoB.

In an aspect, disclosed herein is a system for the production of a bioderived fuel comprising a fermentation vessel that comprises a non-naturally occurring C. autoethanogenum that when provided feedstock comprising carbon monoxide or carbon dioxide from electrolyzer off-gas or flue gas feedstock has improved growth rates and improved alcohol production rates when compared to a naturally occurring C. autoethanogenum; and wherein the bioderived fuel is produced from the alcohol produced by the non-naturally occurring C. autoethanogenum.

Other objects, advantages, and novel features of the present invention will become apparent from the following detailed description of the invention when considered in conjunction with the accompanying drawings.

### DESCRIPTION OF THE DRAWINGS

FIG. 1 depicts a schematic from the initial carbon capture of flue gas to the integration of electrolysis and fermentation to the desired platform chemicals.

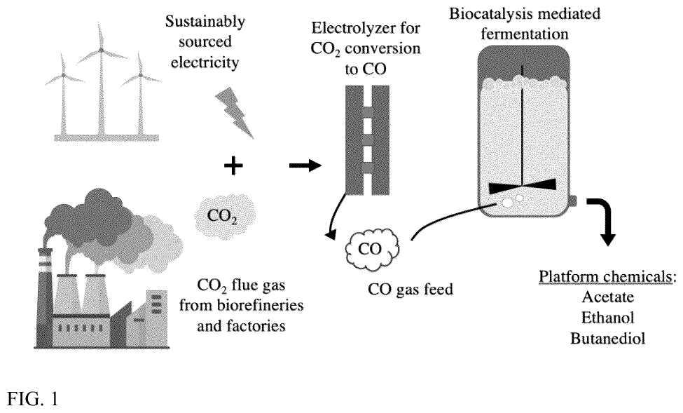

FIG. 2 depicts the concentration of mutagen to percentage of C. autoethanogenum cells killed.

FIG. 3 depicts initial comparison of promising mutants vs the wild type for the high CO ALE.

FIG. 4 depicts a growth curve comparing HB25C8 with the wild type.

FIG. 5 depicts acetic acid production in grams per L that correlates to the growth depicted in FIG. 4.

FIG. 6 depicts ethanol production in grams per L that correlates to the growth depicted in FIG. 4.

FIG. 7 depicts an embodiment of a generic suicide vector design to be deployed for individual gene knock outs.

FIG. 8 depicts a carbon product comparison (ethanol and acetate) of various generated mutant C. autoethanogenum strains as well as wild type C. autoethanogenum.

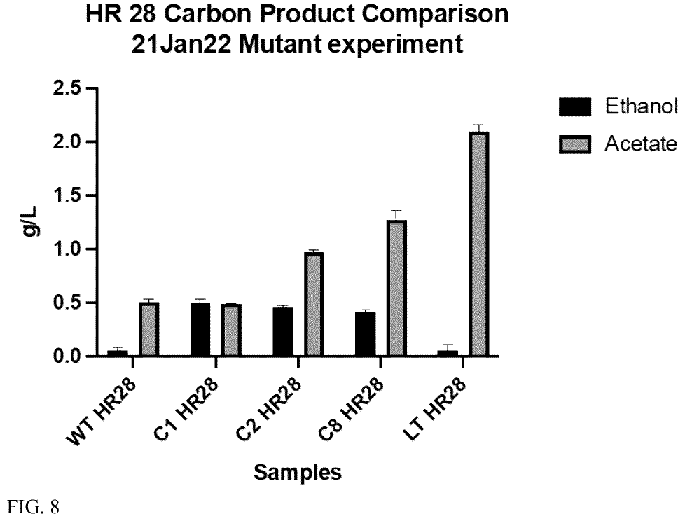

### DETAILED DESCRIPTION

Disclosed herein are non-naturally occurring C. autoethanogenum adapted to electrolyzer off-gas feedstock conditions that possess increased growth rates when compared to naturally occurring naturally occurring C. autoethanogenum.

In an embodiment, disclosed herein are methods for chemical mutagenesis mediated adaptive laboratory evolution (mALE) of C. autoethanogenum under different ratios of CO:H₂ to enhance growth and productivity under variable CO thresholds. In an embodiment, disclosed herein are non-naturally occurring C. autoethanogenum with superior phenotype demonstrating better growth rates than the wild type C. autoethanogenum in batch bottles.

Chemical mutagenesis by way of the mutagen, methylnitronitrosoguanidine (NTG), was deployed in various concentrations to create a kill curve. When the optimal concentration was determined, cells were mutagenized after outgrowth in minimal media (CaMM). The outgrowth from the mutagenesis was then divided into two separate ALE cultures: one with high and low CO concentrations. These two populations were subjected to serial transfers for several months under minimal media and target gas concentration conditions.

After several months of serial transfer, the ALE cells were plated and colonies were picked. Promising isolates were outgrown in competition with the wild type and the top strains were selected for further analysis including: genotyping, metabolite analysis, and further studies to delineate individual mutation impacts including knockouts and transcriptomics.

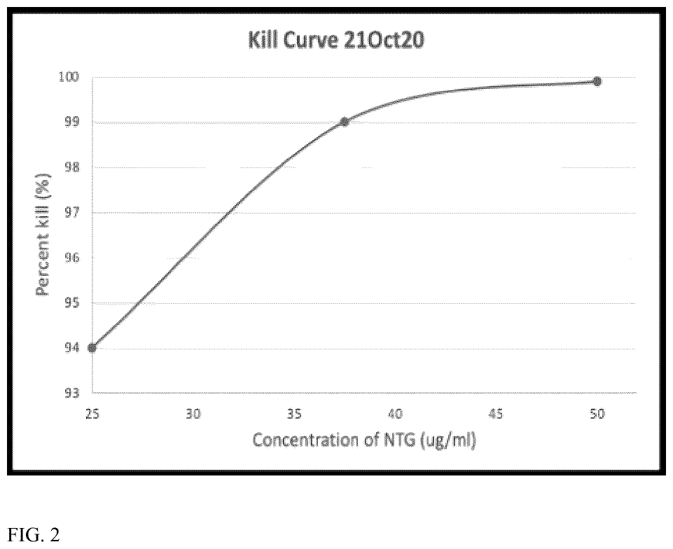

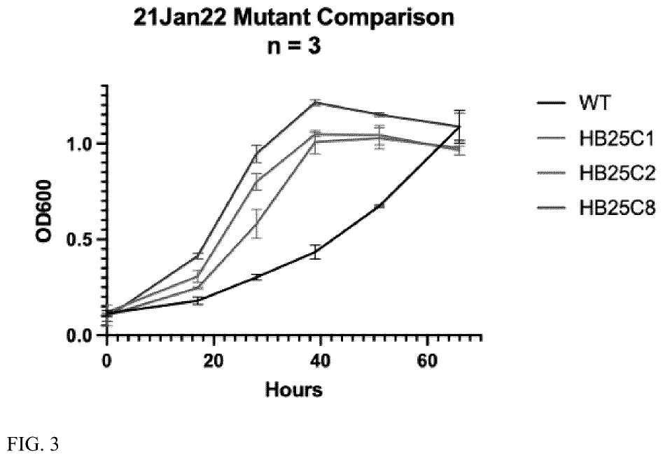

The initial experiment for this project identified the ideal mutagen concentration for full genome coverage while ensuring that all surviving cells had mutations (FIG. 2). It was determined that 25 ug of NTG per mL of C. autoethanogenum cells grown up on minimal media would yield 94% kill. These data allowed us to move into the adaptive evolution phase which lasted several months. Cells from the ALE were plated, scaled up in serum vials, and compared to the wild type (FIG. 3).

Upon identification of potential successful mutants (HB25C8 from FIG. 3), it was determined to genotype and analyze possible mutations that were influencing this phenotype. The genotyping of our top strain revealed nearly 50 mutations. In order to make data analysis more manageable, we determined to categorize the mutations to better analyze their impacts. These trends are summarized in Table 1.

TABLE 1
summarizes the mutation profile of HB25C8 and quantifies
different classes of mutations.

| Number | |
|---|---|
| of Genes | Type of mutation or class of genes impacted |
| 4 | Early termination by a stop codon or a frame shift |
| 1 | Operon deletion (containing 3 genes) |
| 7 | Transcriptional regulators and response regulator-related |
| | gene mutations |
| | (Three of these genes are also the genes with early |
| | termination by stop codon/frame shift) |
| 5 | Promoter region mutations impacting potentially six |
| | different genes |
| | (One of these is a promoter for a transcriptional regulator) |
| 2 | Sporulation pathway genes |
| 14 | Hypothetical protein annotations with known domains |
| | but undefined functions |
| 4 | Codon changes with no amino acid sequence change |

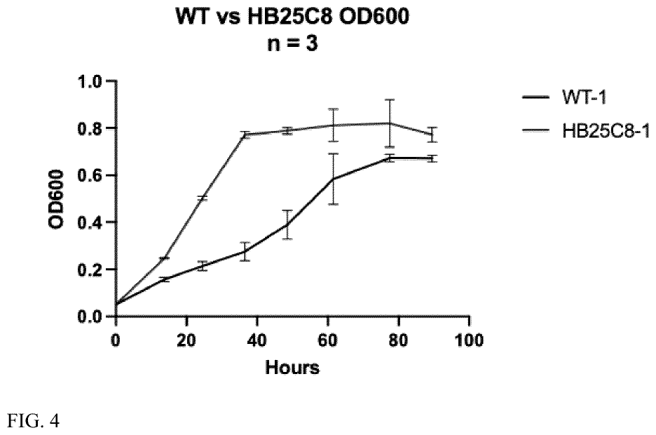

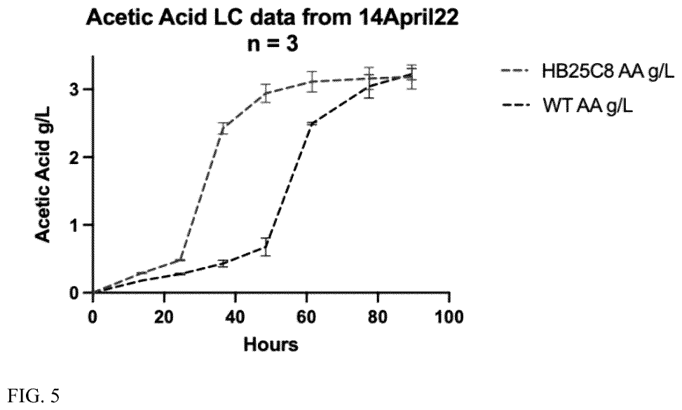

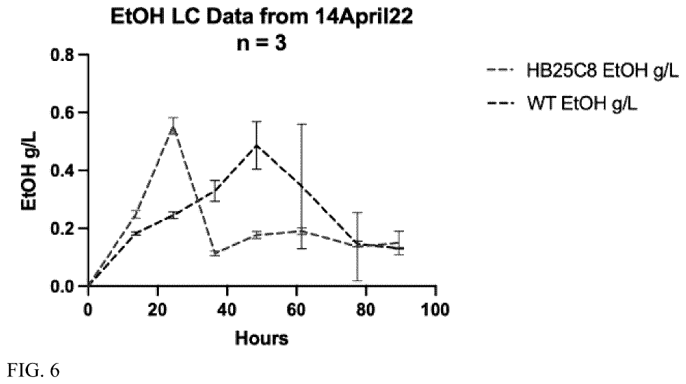

After analyzing the mutation profile, it was determined to repeat the growth curve comparison with the wild type and perform metabolite analysis for target products in serum vials to ensure product yield was comparable or better than the wild type. These findings are summarized in FIGS. 4-6.

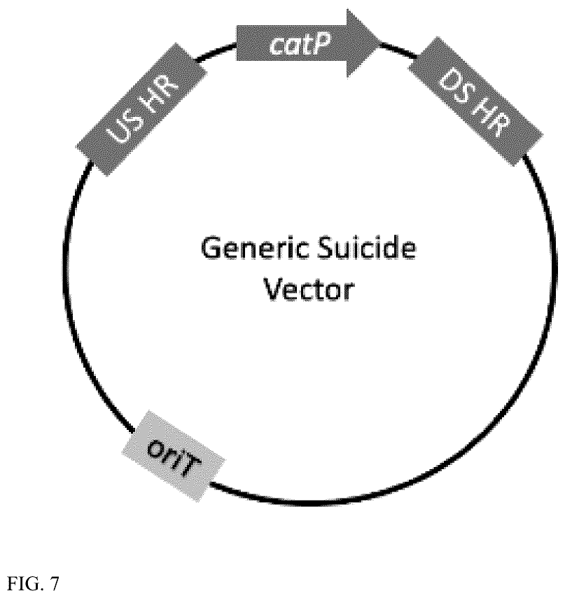

We have designed several experiments to more fully explore these mutations. Since our mutation profile had seven genes that had early termination or complete gene deletions, we have designed suicide vectors with homology regions to knockout each of these genes individually (FIG. 7) and verify their individual impact on the phenotype as compared to the wild type and HB25C8. In an embodiment, the mutations in a non-naturally occurring C. autoethanogenum adapted to electrolyzer off-gas feedstock conditions that possess increased growth rates when compared to naturally occurring naturally occurring C. autoethanogenum include, but are not limited to, a stop codon in the Crp/Fnr family transcriptional regulator; a stop codon in the xanthine dehydrogenase family protein molybdopterin-binding subunit; a stop codon in the sporulation transcription factor Spo0A, a deletion of about 400 nt bp in nusB (a transcription antitermination factor); and an operon deletion of about 2.7 Kpb in deoB and/or CAETHG_RS19245. Additionally, since so many of the mutations observed impact transcription, we are awaiting data from a transcriptomic study for log and stationary phase expression in the wild type and HB25C8. A second set of microbial physiology experiments is being performed on the low CO ALE mutant population to determine if there are promising mutants for those conditions. Finally, all top mutant or high-performing individual knockout strains will need to be tested in fermenter-scale experiments to verify they out-perform the wild type at scale. In an embodiment, an electrolysis apparatus electrolyzer and fermentation system is used in tandem with a wild type outgrowth.

In conclusion, the chemical mutagenesis/ALE process we deployed for strain enhancement has generated a number of mutants that outperform the wild type. The top mutant we tested had shorter lag times and higher biomass accumulation under an electrolyzer's high CO production conditions. We have preliminary data to suggest we may also have enhanced low CO mutants as well. If we can verify these strains maintain their enhanced performance at scale, this has significant positive implications for industrial applications.

The foregoing discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. The following claims are hereby incorporated into this Detailed Description, with each claim standing on its own as a separate aspect, embodiment, or configuration.

What is claimed is:

1. A non-naturally occurring C. autoethanogenum that when provided feedstock comprising carbon monoxide or carbon dioxide from electrolyzer off-gas or flue gas feedstock has improved growth rates and improved alcohol or acetate production rates when compared to a naturally occurring C. autoethanogenum.

2. The non-naturally occurring C. autoethanogenum of claim 1 comprising a mutation in at least one gene encoding for a Crp/Fnr family transcriptional regulator.

3. The non-naturally occurring C. autoethanogenum of claim 2 comprising a mutation that creates a stop codon in a Crp/Fnr family transcriptional regulator.

4. The non-naturally occurring C. autoethanogenum of claim 1 comprising a mutation in at least one gene encoding for a xanthine dehydrogenase family protein molybdopterin-binding subunit.

5. The non-naturally occurring C. autoethanogenum of claim 1 comprising a mutation in at least one gene encoding for a sporulation transcription factor.

6. The non-naturally occurring C. autoethanogenum of claim 5 wherein the mutation is in sporulation transcription factor SpoOA.

7. The non-naturally occurring C. autoethanogenum of claim 1 comprising a mutation in at least one gene encoding for a transcription antitermination factor.

8. The non-naturally occurring C. autoethanogenum of claim 7 wherein the mutation comprises a deletion of about 400 bp.

9. The non-naturally occurring C. autoethanogenum of claim 7 wherein the mutation is in a nusB gene.

10. The non-naturally occurring C. autoethanogenum of claim 1 comprising a deletion of about 2.7 Kpb in deoB.

11. The non-naturally occurring C. autoethanogenum of claim 1 comprising a deletion of about 2.7 Kpb in CAETHG_RS19245.

12. The non-naturally occurring C. autoethanogenum of claim 1 wherein the alcohol is ethanol.

13. A method for making a bioderived fuel comprising providing a non-naturally occurring C. autoethanogenum feedstock comprising carbon monoxide or carbon dioxide from electrolyzer off-gas or flue gas feedstock and isolating an alcohol that is used to make the bioderived fuel.

14. The method of claim 13 wherein the non-naturally occurring C. autoethanogenum comprises a mutation in at least one gene encoding for a xanthine dehydrogenase family protein molybdopterin-binding subunit.

15. The method of claim 13 wherein the non-naturally occurring C. autoethanogenum comprises a mutation in at least one gene encoding for a Crp/Fnr family transcriptional regulator.

16. The method of claim 13 wherein the non-naturally occurring C. autoethanogenum comprises a mutation in at least one gene encoding for a sporulation transcription factor.

17. The method of claim 13 wherein the non-naturally occurring C. autoethanogenum comprises a mutation in at least one gene encoding for a transcription antitermination factor.

18. The method of claim 17 wherein the non-naturally occurring C. autoethanogenum comprises a mutation in a nusB gene.

19. The method of claim 13 wherein the non-naturally occurring C. autoethanogenum comprises a deletion of about 2.7 Kpb in deoB.

20. A system for the production of a bioderived fuel comprising a fermentation vessel that comprises a non-naturally occurring C. autoethanogenum that when provided feedstock comprising carbon monoxide or carbon dioxide from electrolyzer off-gas or flue gas feedstock has improved growth rates and improved alcohol production rates when compared to a naturally occurring C. autoethanogenum; and wherein the bioderived fuel is produced from the alcohol produced by the non-naturally occurring C. autoethanogenum.
